SEQUENCE LISTING (1 sequence)

SEQ ID NO: 1 - DNA, 75 bp, Borrelia burgdorferi
atgaagaaggatgatcagattgctgctgctattgctttgagggggatggctaaggatggaaagtttgctgtgaag

(19) United States
(12) Patent Application Publication
(10) Pub. No.: US 2022/0244251 A1
(43) Pub. Date: Aug. 4, 2022

(54) Targets and Methods of Diagnosing and Monitoring Lyme Disease

(71) Applicant: Board of regents of the Nevada System of Higher Education on Behalf of the University of Nevada Reno, Reno, NV (US)

(72) Inventors: David AUCOIN, Reno, NV (US); Kathryn J. PFLUGHOEFT, Reno, NV (US)

(21) Appl. No.: 17/614,168

(22) PCT Filed: May 27, 2020
(86) PCT No.: PCT/US2020/034679

§ 371 Date: Nov. 24, 2021

Related U.S. Application Data
(60) Provisional application No. 62/854,272, filed on May 29, 2019.

(51) Int. Cl. G01N 33/564

(52) U.S. Cl. CPC G01N 33/564

(57) ABSTRACT

Disclosed herein is a method of detecting and identifying antigens that are shed into human bodily fluids during infection. The disclosed method allows circulating antigens associated with a particular infection to be detected within minutes or hours from testing as compared to days required with the current methods. Methods of identifying diagnostic indicators/targets for a given condition or disease are disclosed which include immunizing a veterinary subject with biological fluids obtained from a human infected with particular antigens to identify diagnostic targets for immunoassay. Also disclosed are methods of diagnosing and monitoring a B. burgdorferi-associated condition, such as Lyme disease. Point-of-care immunoassays are also provided that can be used to diagnose or monitor the efficacy of a B. burgdorferi-associated condition treatment. These immunoassays can also be used for rapid diagnosis of infection produced by B. burgdorferi, such as Lyme disease.

### CROSS REFERENCE FOR RELATED APPLICATION

This disclosure claims priority to U.S. Provisional Patent No. 62/854,272 filed on May 29, 2019, which is hereby incorporated by reference in its entirety.

### ACKNOWLEDGMENT OF GOVERNMENT SUPPORT

This invention was made with government support under Grant Nos. R41AI114049 and GM103440 awarded by the National Institutes of Health. The government has certain rights in the invention.

### FIELD

This disclosure relates to antigen detection and specifically to detecting and identifying antigens circulating in human biological samples for diagnosing and monitoring conditions, including detecting and identifying antigens secreted/shed by Borrelia burgdorferi for diagnosing and monitoring Lyme disease.

### BACKGROUND

Early diagnosis is critical for treatment of an infection to be effective. Diagnostic assays that are capable of detecting low levels of a particular molecule, such as an antigen, could greatly impact patient outcome because they would be able to detect the molecule and thus a condition associated with such within minutes or hours from testing as compared to days required with the current methods. Earlier detection translates into earlier administration of therapies, which could significantly increase the likelihood of patient survival as well as decrease the severity of the disease.

Current diagnostic tools are limited and diagnosis with these methods often occurs when the infection is so severe that treatment is inefficient and ineffective. For example, diagnosing infections, such as bacterial and fungal infections, is often plagued by symptoms of the particular infection being non-specific making it difficult to obtain an accurate diagnosis at the onset of the disease. Current diagnostic assays often can only detect a particular molecule, such as an antigen, associated with a particular disease or condition if such molecule is present at high levels, thus only detecting the infection associated with the particular molecule not until the infection is well developed.

### SUMMARY

Disclosed herein is a multi-platform strategy to assess microbial biomarkers that can be consistently detected in host samples, using Borrelia burgdorferi, the causative agent of Lyme disease, as an example. Key aspects of the strategy include the selection of a macaque model of human disease, In vivo Microbial Antigen Discovery (InMAD), and proteomic methods that include microbial biomarker enrichment within samples to identify secreted proteins circulating during infection. Using the described strategy, the inventors identified 6 biomarkers from multiple samples. In addition, the temporal antibody response to select bacterial antigens was mapped. By integrating biomarkers identified from early infection with temporal patterns of expression, the described platform allows for the data driven selection of diagnostic targets.

Based on these findings, disclosed herein are methods of identifying diagnostic indicators. In some embodiments, these methods include selecting a condition or disease for which a diagnostic assay is desired and is believed to be associated with one or more antigens; immunizing a veterinary subject which is not afflicted with the selected condition or disease with a human biological sample obtained from a human subject having the selected condition or disease; detecting one or more antigens in a biological sample obtained from the immunized animal subject; comparing the one or more antigens detected in the immunized animal subject sample with a control; and identifying one or more diagnostic indicators for the selected condition or disease, wherein an alteration in at least one antigen detected in the sample obtained from the immunized subject relative to the control indicates that such antigen is a diagnostic indicator for the condition or disease.

In some embodiments, the method further includes obtaining the biological sample, such as serum or urine, from the human subject with the selected condition or disease.

In some embodiments, the method further includes filtering the human biological sample obtained from the human subject to isolate the one or more soluble antigens.

In some embodiments, the method further includes obtaining the biological sample, such as serum or urine, from the immunized animal subject prior to detecting one or more antigens.

In some embodiments of the method, detecting one or more antigens in a biological sample obtained from the immunized animal subject includes using one-dimensional or two-dimensional immunoblots followed by mass spectroscopy to identify the one or more antigens.

In some embodiments, methods are provided for diagnosing and monitoring an antigen-associated condition, such as Borrelia burgdorferi-associated condition including Lyme disease. In one example, the disclosed methods allow for self-monitoring in which a subject, such as an immunosuppressed patient, monitors the presence of one or more specific antigens, to monitor the onset of an infection.

The foregoing and other features and advantages of the disclosure will become more apparent from the following detailed description, which proceeds with reference to the accompanying figures.

### BRIEF DESCRIPTION OF DRAWINGS

FIG. 1 is a schematic for Multiplatform Approach for Microbial Biomarker Identification—Microbial biomarkers were directly or indirectly identified from samples collected from an infected host, in the case of this study, a macaque model of infection. Techniques used for direct detection of microbial biomarkers included mass spectrometry (MS) of concentrated or enriched samples and protein array. Indirect detection, included the InMAD strategy coupled with protein array and immunoprecipitation-coupled MS. Identified biomarkers were categorized based upon the number of times each was identified by either direct or indirect analysis.

FIG. 2 shows a time course of infection and sample collection. Rhesus macaques were infected with B. burgdorferi using a natural tick-bite model of Lyme disease. Blood, urine, and cerebrospinal fluid were collected throughout the 4-month infection.

FIG. 3 shows a serological response to a natural B. burgdorferi infection using a 5-antigen multiplex Luminee-based assay. Each graph represents one animal, with the antigens detected distinguished by color. Note, only KD91, KC92, and KG87 were assessed at weeks 2 and 3. Vertical axis: MFI=mean fluorescence intensity. Shown is the mean±SEM for each time point. The mean values obtained from pre-immune serum of each individual macaque was subtracted from the MFI for each time point.

FIGS. 4A-4B illustrate dynamics of immunogenic response of macaques to B. burgdorferi as assessed by a limited NAPPA array. FIG. 4A. Normalized signal intensities across the array were calculated by subtracting the background individual spot intensity of negative controls from the individual spot intensity. This is divided by the median array spot intensity minus the background spot intensity. Typically, a minimal signal-to-noise ratio of 1.4 provides detectable signals in ELISA validation assays. Serum used to probe the array was collected at 0-14 weeks post-infection (TO-T14). FIG. 4B. A portion of the limited array is provided representing the temporal response (week post-infection, TO-T14) of macaque KD89 to 6 Bb proteins, 5 negative controls, and 1 positive control (boxed). Each protein is represented by 3 spots on the array.

### DETAILED DESCRIPTION OF SEVERAL EMBODIMENTS

This technology disclosed herein is described in one or more exemplary embodiments in the following description with reference to the Figures. Reference throughout this specification to “one embodiment,” “an embodiment,” or similar language means that a particular feature, structure, or characteristic described in connection with the embodiment is included in at least one embodiment of the present technology disclosed herein. Thus, appearances of the phrases “in one embodiment,” “in an embodiment,” and similar language throughout this specification may, but do not necessarily, all refer to the same embodiment.

The described features, structures, or characteristics of the technology disclosed herein may be combined in any suitable manner in one or more embodiments. In the following description, numerous specific details are recited to provide a thorough understanding of embodiments of the technology disclosed herein. One skilled in the relevant art will recognize, however, that the technology disclosed herein may be practiced without one or more of the specific details, or with other methods, components, materials, and so forth. In other instances, well-known structures, materials, or operations are not shown or described in detail to avoid obscuring aspects of the technology disclosed herein.

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

As used herein, “one or more” or at least one can mean one, two, three, four, five, six, seven, eight, nine, ten or more, up to any number.

As used herein, the term “comprises” means “includes.” Hence “comprising A or B” means including A, B, or A and B. It is further to be understood that all base sizes and all molecular weight or molecular mass values given for peptides and nucleic acids are approximate and are provided for description.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing of this disclosure are described below. Such methods and materials are illustrative only and are not intended to be limiting. Although methods and materials similar or equivalent to those described herein can be used in the practice or testing of the present disclosure, suitable methods and materials are described below. All publications, patent applications, patents, and other references mentioned herein are incorporated by reference in their entirety. Unless otherwise defined, all technical terms used herein have the same meaning as commonly understood. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. In addition, the materials, methods, and examples are illustrative only and not intended to be limiting.

In order to facilitate review of the various embodiments of this disclosure, the following explanations of specific terms are provided:

Alteration or difference: An increase or decrease in the amount of something, such as a protein antigen. In some examples, the difference is relative to a control or reference value or range of values, such as an amount of a protein that is expected in a subject who does not have a particular condition or disease being evaluated. Detecting an alteration or differential expression/activity can include measuring a change in protein expression, concentration or activity, such as by ELISA, Western blot and/or mass spectrometry. For example, an alteration can be an increase in expression (up-regulation) or a decrease in expression (down-regulation). In some examples, the difference is relative to a control or reference value, such as an amount of expression in a sample from a healthy control subject.

Animal: Living multi-cellular vertebrate organisms, a category that includes, for example, mammals and birds. The term mammal includes both human and non-human mammals. Similarly, the term “subject” includes both human and veterinary subjects, for example, mice.

Antibody: A polypeptide ligand comprising at least a light chain or heavy chain immunoglobulin variable region which specifically binds an epitope of a protein listed in the tables below, or a fragment of any of these proteins. Antibodies can include a heavy chain and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric or humanized antibodies that may be derived from a murine antibody, heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. An “antigenic polypeptide” is a polypeptide to which an immune response, such as a T cell response or an antibody response, can be stimulated. “Epitope” or “antigenic determinant” refers to a site on an antigen to which B and/or T cells respond. Methods of determining spatial conformation of epitopes include, for example, x-ray crystallography and multi-dimensional nuclear magnetic resonance spectroscopy. The term “antigen” denotes both subunit antigens, (for example, antigens which are separate and discrete from a whole organism with which the antigen is associated in nature), as well as killed, attenuated or inactivated bacteria, viruses, fungi, parasites or other microbes. An “antigen,” when referring to a protein, includes a protein with modifications, such as deletions, additions and substitutions (generally conservative in nature) to the native sequence, so long as the protein maintains the ability to elicit an immunological response, as defined herein. These modifications may be deliberate, as through site-directed mutagenesis, or may be accidental, such as through mutations of hosts which produce the antigens.

Bacteria: A large domain of prokaryotic microorganisms. Typically, a few micrometers in length, bacteria have a wide range of shapes, ranging from spheres to rods and spirals. There are broadly speaking two different types of cell wall in bacteria, called Gram-positive and Gram-negative. Gram-positive bacteria possess a thick cell wall containing many layers of peptidoglycan and teichoic acids. In contrast, Gram-negative bacteria have a relatively thin cell wall consisting of a few layers of peptidoglycan surrounded by a second lipid membrane containing lipopolysaccharides and lipoproteins. Most bacteria have the Gram-negative cell wall, and only the Firmicutes and Actinobacteria have the alternative Gram-positive arrangement.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Biological sample: A biological specimen containing genomic DNA, RNA (such as mRNA), protein, or combinations thereof, obtained from a subject. Examples include, but are not limited to, saliva, peripheral blood, urine, tissue biopsy, surgical specimen, and autopsy material. In embodiments, the biological sample is a bodily fluid, such as blood, or a component thereof, such as plasma or serum.

Biomarker: Molecular, biological or physical attributes that characterize a physiological state and can be objectively measured to detect or define disease progression or predict or quantify therapeutic responses. For instance, a substance used as an indicator of a biologic state. It is a characteristic that is objectively measured and evaluated as an indicator of normal biologic processes, pathogenic processes, or pharmacologic responses to a therapeutic intervention.

Borrelia burgdorferi: A gram-negative bacteria. A “Borrelia burgdorferi-associated molecule” is a molecule associated with one or more signs or symptoms of Lyme disease. In some examples, a Borrelia burgdorferi-associated molecule is one or more of the antigens disclosed herein.

Contacting: “Contacting” includes in solution and solid phase. “Contacting” can occur in vitro with, e.g., samples, such as biological samples containing a target biomolecule, such as an antibody. “Contacting” can also occur in vivo.

Diagnosis: The process of identifying a condition or disease by its signs, symptoms, results of various tests and presence of diagnostic indicators. The conclusion reached through that process is also called “a diagnosis.” Forms of testing commonly performed include blood tests, medical imaging, genetic analysis, urinalysis, biopsy and the methods disclosed herein.

Diagnostically significant amount: As used herein a “diagnostically significant amount” refers to an increase or decrease in the level of a gene product, such as a protein or ratio thereof in a biological sample that is sufficient to allow one to distinguish one patient population from another. In some embodiments, the diagnostically significant increase or decrease is at least 2-fold, at least 3-fold, at least 4-fold, at least 5-fold, at least 6-fold, at least 8-fold, at least 10-fold, at least 15-fold, at least 20-fold, at least 30-fold or at least 40-fold relative to a control. In some embodiments, the diagnostically significant increase or decrease is at least 2-fold, at least 3-fold, at least 4-fold, at least 5-fold, at least 6-fold, at least 8-fold, at least 10-fold, at least 15-fold, at least 20-fold, at least 30-fold or at least 40-fold change in the ratio of two or more biomarkers relative to a control.

Immunoassay: A biochemical test that measures the presence or concentration of a substance in a sample, such as a biological sample, using the reaction of an antibody to its cognate antigen, for example the specific binding of an antibody to a protein. Both the presence of antigen and the amount of antigen present can be measured. For measuring proteins, for each the antigen and the presence and amount (abundance) of the protein can be determined or measured. Measuring the quantity of antigen can be achieved by a variety of methods. One of the most common is to label either the antigen or antibody with a detectable label.

An “enzyme linked immunosorbent assay (ELISA)” is type of immunoassay used to test for antigens (for example, proteins present in a sample, such as a biological sample). A “competitive radioimmunoassay (RIA)” is another type of immunoassay used to test for antigens. A “lateral flow immunochromatographic (LFI)” assay is another type of immunoassay used to test for antigens.

Increase or upregulate: To enhance the quality, amount, or strength of something. In one example, an agent increases the activity or expression of a molecule disclosed herein, for example relative to an absence of the agent. In some examples, an increase in expression refers to an increase in a disclosed gene product or activity of a disclosed gene product. A gene product can be RNA (such as mRNA, rRNA, tRNA, and structural RNA) or protein.

Gene upregulation includes any detectable increase in the production of a gene product. In certain examples, production of a gene product increases by at least 2-fold, for example at least 3-fold or at least 4-fold as a result of a specific condition or disease as compared to a control (such an amount of gene expression in a sample of a subject that is not afflicted with the condition or disease). Such increases can be measured using the methods disclosed herein. For example, “detecting or measuring expression of a disclosed molecule” includes quantifying the amount of the gene, gene product or modulator thereof present in a sample. Quantification can be either numerical or relative. Detecting expression of the gene, gene product or modulators thereof can be achieved using any method known in the art or described herein, such as by PCR (such as quantitative RT-PCR), ELISA, Western blot or mass spectrometry. In primary embodiments, the change detected is an increase or decrease in expression as compared to a control, such as a biological sample or subject that has not been exposed or contacted with a therapeutic agent. In some examples, the detected increase or decrease is an increase or decrease of at least two-fold compared with the control or standard. In other embodiments of the methods, the increase or decrease is of a diagnostically significant amount.

Label: A detectable compound or composition that is conjugated directly or indirectly to another molecule, such as an antibody or a protein, to facilitate detection of that molecule. Specific, non-limiting examples of labels include fluorescent tags, enzymatic linkages (such as horseradish peroxidase), radioactive isotopes (for example ¹⁴C, ³²P, ¹²⁵I, ³H isotopes and the like) and particles such as colloidal gold. In some examples a protein, such as a protein associated with a particular infection, is labeled with a radioactive isotope, such as ¹⁴C, ³²P ¹²⁵I, ³H isotope. In some examples an antibody that specifically binds the protein is labeled. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998), Harlow & Lane (Antibodies, A Laboratory Manual, Cold Spring Harbor Publications, New York, 1988).

Lyme Disease: An infectious disease caused by a Gram-negative bacterium, Borrelia burgdorferi transmitted to humans through the bite of infected blacklegged ticks. Typical symptoms include fever, headache, fatigue, and a characteristic skin rash called erythema migrans. If left untreated, infection can spread to joints, the heart, and the nervous system. Lyme disease is diagnosed based on symptoms, physical findings (e.g., rash), and the possibility of exposure to infected ticks. Laboratory testing is helpful if used correctly and performed with validated methods. Most cases of Lyme disease can be treated successfully with a few weeks of antibiotics. Steps to prevent Lyme disease include using insect repellent, removing ticks promptly, applying pesticides, and reducing tick habitat. The ticks that transmit Lyme disease can occasionally transmit other tickborne diseases as well.

The methods, compositions and assays disclosed herein provide a means of identifying a subject who has Lyme disease or who is at increased risk of developing Lyme disease. A “non-Lyme disease” or “normal” subject does not have any form of Lyme disease.

A “Lyme disease-associated molecule” is a molecule associated with one or more signs or symptoms of Lyme disease. In some examples, a Lyme disease-associated molecule is one or more of the antigens disclosed herein.

Microorganism: A single-celled, or unicellular, organism which include bacteria, fungi, archaea or protists, but not viruses and prions (which are generally classified as non-living). Microorganisms that cause disease in a host are known as pathogens.

Under conditions sufficient to: A phrase that is used to describe any environment that permits the desired activity. In some examples, under conditions sufficient to includes suitable conditions for binding of peptides-antibody on the array and/or any of the in vitro assays.

II. Methods for Detecting and Identifying Circulating Antigens

Disclosed herein are methods for detecting and identifying circulating antigens that can be used to identify diagnostic indicators/targets of specific conditions and/or diseases. In one example, a method of identifying one or more diagnostic indicators includes selecting a condition or disease for which a diagnostic assay is desired and is believed to be associated with one or more antigens. For example, the condition can be one that is associated with a particular set of clinical factors/symptoms or presence of a microorganism such a bacteria.

The method for identifying one or more diagnostic indicators also includes immunizing a verterinary subject (such as a mouse or rabbit) that is not afflicted with the selected condition or disease with a human biological sample obtained from a human subject having the selected condition or disease. For example, a biological sample, such as urine, is collected from a human subject displaying one or more signs or symptoms associated with the selected condition or disease for which a diagnostic assay is desired. In other examples, other biological fluids, such as blood (such as whole blood obtained from a finger prick), GCF, amniotic fluid, BALF, salvia or tears are collected. In some embodiments, the method further includes filtering the human biological sample obtained from the human subject to isolate the one or more soluble antigens present in the sample.

The disclosed method for identifying one or more diagnostic indicators/targets also includes detecting one or more antigens in a biological sample obtained from the immunized animal subject; comparing the one or more antigens detected in the immunized animal subject sample with a control; and identifying one or more diagnostic indicators for the selected condition or disease, wherein an alteration in at least one antigen detected in the sample obtained from the immunized subject relative to the control indicates that such antigen is a diagnostic indicator for the condition or disease. In some examples, the method further includes obtaining the biological sample, such as serum or urine, from the immunized animal subject prior to detecting one or more antigens. In some embodiments of the method, detecting one or more antigens in a biological sample obtained from the immunized animal subject includes using one-dimensional or two-dimensional immunoblots followed by mass spectroscopy to identify the one or more antigens.

In some examples, the method includes detecting an increase, such as a statistically significant increase, such as an at least a 1.5, 2, 3, 4, or 5 fold increase in the amount of one or more molecules associated with condition or disease, including at least a 1.5, 2, 3, 4, or 5 fold increase to a control or reference value, such as between a 1.5 to 5 fold increase, a 2 to 6 fold increase, a 3 to 10 fold increase, including a 2 fold, a 3 fold, a 4 fold, a 5 fold, a 6 fold, a 7 fold, a 8 fold, a 9 fold or 10 fold increase. In some embodiments, the method includes detecting a decrease, such as a statistically significant decrease, such as at least a 2, 3, 4, or 5 fold decrease in the amount of one or more molecules associated with the selected condition or disease, such as one or more protein antigens, as compared to a control or reference sample, such as between a 1.5 to 5 fold decrease, a 2 to 6 fold decrease, a 3 to 10 fold decrease, including a 2 fold, a 3 fold, a 4 fold, a 5 fold, a 6 fold, a 7 fold, a 8 fold, a 9 fold or 10 fold decrease.

In some embodiments of the method, the disclosed methods allow for self-monitoring in which a subject, such as an immunosuppressed patient, monitors the presence of one or more specific antigens, to monitor the onset of an infection.

Methods for Detecting B. burgdorferi-Associated Condition and Monitoring the Efficacy of a Therapeutic Regimen

Methods are disclosed herein that are of use to determine if a subject has a B. burgdorferi-associated condition, such as Lyme disease, or to monitor the efficacy of therapy. These methods utilize a biological fluid, such as, but not limited to urine or serum, for the detection of a molecule associated B. burgdorferi, such as Lyme disease, including, but not limited to, protein antigens disclosed herein including those listed in Table 3. The B. burgdorferi-associated molecules, such as Lyme disease-associated molecules, include any naturally occurring forms of the proteins, such as but not limited to glycosylated forms. These methods can be performed over time, to monitor the progression or regression of Lyme disease in a subject, or to assess for the development of Lyme disease from a pre-Lyme disease condition. In additional examples, the disclosed methods and kits are used for self monitoring in which a subject, such as a subject that has previously been diagnosed and treated for a Lyme disease associated condition or disease practices the method or uses the kit to monitor for relapse.

Methods are disclosed herein that include testing a biological sample, such as a serum or urine sample, obtained from a human at risk or suspected of having Lyme disease. In one example, the biological sample is a biological fluid, such as urine. However, other biological fluids are also of use, such as blood (such as whole blood obtained from a finger prick), GCF, amniotic fluid, BALF, salvia or tears. The methods include detecting, or determining the abundance (amount) of one or more molecules associated with Lyme disease, including protein antigens listed in Table 1. In some examples, the methods include determining a proteomic profile.

In one example, the method includes detecting at least one more molecule associated with Lyme disease such as one or more molecules listed in Table 3. The methods can include detecting at least one, such as at least two, at least three, at least four, at least five, at least six, including one, two, three, four, five, or six molecules associated with Lyme disease. In one example, the method includes detecting at least one, at least two, at least three, at least four, at least five, at least six, including one, two, three, four, five, or six molecules listed in Table 3.

In some embodiments, the method includes detecting an increase, such as a statistically significant increase, such as an at least a 1.5, 2, 3, 4, or 5 fold increase in the amount of one or more molecules associated with Lyme disease, including at least a 1.5, 2, 3, 4, or 5 fold increase in one or more protein antigens listed in Table 3 as compared to a reference value. In some embodiments, the method includes detecting a decrease, such as a statistically significant decrease, such as at least a 2, 3, 4, or 5 fold decrease in the amount of one or more protein antigens listed in Table 3 as compared to a reference sample.

In one embodiment, the method includes comparing a proteomic profile of a test sample of urine from a human subject of interest comprising at least one of protein associated with Lyme disease, such a protein antigen listed in Table 3, with a proteomic profile from a reference sample. In one embodiment, the method determines if the human subject has Lyme disease. If the reference sample is a normal sample and the proteomic profile of the test sample is essentially the same as the proteomic profile of the normal sample, the human subject is determined not to have Lyme disease. However, if the proteomic profile of the test sample has a unique expression signature relative to the proteomic profile of the normal sample the human subject is determined to have Lyme disease.

In some embodiments, if the reference sample is a sample from a human subject with Lyme disease, and its proteomic profile shares at least one unique expression signature characteristic with the reference sample, then the human subject is determined to have Lyme disease. If the proteomic profile of the test sample has a unique expression signature relative to the reference sample the human subject is determined not to have Lyme disease. Hence, the proteomic profile provides an additional diagnostic criterion for these disorders.

In one embodiment, the method is a method to determine if a therapy is effective for the treatment of the human subject by detecting the presence of at least one protein associated with Lyme disease. The method can be performed multiple times over a specified time period, such as days, weeks, months or years. In several examples, the therapy includes treatment with a therapeutic agent for Lyme disease. If the reference sample is a normal human sample, and the proteomic profile of the test sample is essentially the same as the proteomic profile of the normal sample the human subject is determined to have an effective therapy, while if the proteomic profile of the test sample has a unique expression signature relative to the proteomic profile of the normal sample to have an ineffective therapy. If the reference sample is a sample from a human subject with Lyme disease, and proteomic profile shares at least one unique expression signature characteristic with the reference sample then the human subject is determined to have an ineffective therapy, while if the proteomic profile of the test sample has a unique expression signature relative to the reference sample the human subject is determined to have an effective therapy. Changes in the profile can also represent the progression (or regression) of the disease process. Methods for monitoring the efficacy of therapeutic agents are described below.

Monitoring

The diagnostic methods of the present disclosure are valuable tools for practicing physicians to make quick treatment decisions for Lyme disease conditions, including both acute and chronic Lyme disease. These treatment decisions can include the administration of an anti-Lyme disease agent and decisions to monitor a subject for onset and/or advancement of Lyme disease. The method disclosed herein can also be used to monitor the effectiveness of a therapy.

Following the measurement of the expression levels of one or more of the molecules identified herein, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. Based on the measurement, the therapy administered to a subject can be modified.

In one embodiment, a diagnosis, prediction and/or treatment recommendation based on the expression level in a test subject of one or more of the Lyme disease associated molecules disclosed herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

In several embodiments, identification of a subject as having Lyme disease results in the physician treating the subject, such as prescribing one or more therapeutic agents for inhibiting or delaying one or more signs and symptoms associated with Lyme disease. In additional embodiments, the dose or dosing regimen is modified based on the information obtained using the methods disclosed herein.

The subject can be monitored while undergoing treatment using the methods described herein in order to assess the efficacy of the treatment protocol. In this manner, the length of time or the amount give to the subject can be modified based on the results obtained using the methods disclosed herein.

Immunoassays for Diagnosing and Monitoring B. burgdorferi-Associated Conditions

The methods disclosed herein can be performed in the form of various immunoassay formats, which are well known in the art. There are two main types of immunoassays, homogeneous and heterogeneous. In homogeneous immunoassays, both the immunological reaction between an antigen and an antibody and the detection are carried out in a homogeneous reaction. Heterogeneous immunoassays include at least one separation step, which allows the differentiation of reaction products from unreacted reagents. A variety of immunoassays can be used to detect one or more of the molecules capable of detecting a B. burgdorferi-associated molecule, including detecting extracellular polysaccharides. In one example, one or more antigens associated with an B. burgdorferi-associated disorder/condition are measured to diagnose an B. burgdorferi-associated disorder, such as Lyme disease. For example, one or more antigens listed in Table 3 are detected with a disclosed immunoassay. In some examples, the disclosed immunoassay includes at least one, such as two, three, four, five, six, or more molecules associated with a B. burgdorferi-associated condition or disease, such as Lyme disease. In one example, the immunoassay includes at least one, such as two, three, four, five, or six molecules listed in Table 3.

ELISA is a heterogeneous immunoassay, which has been widely used in laboratory practice since the early 1970s, and can be used in the methods disclosed herein. The assay can be used to detect protein antigens in various formats. In the “sandwich” format the antigen being assayed is held between two different antibodies. In this method, a solid surface is first coated with a solid phase antibody. The test sample, containing the antigen (e.g., a diagnostic protein), or a composition containing the antigen, such as a urine sample from a subject of interest, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. The amount of color intensity resulting from the enzyme-substrate interaction is a measure of the amount of antigen in the sample tested. A heterogeneous immunoassay, such as an ELISA, can be used to detect any molecules associated with a B. burgdorferi antigen.

In another example, immuno-PCR can be used to detect any of the molecules associated with a B. burgdorferi condition such as Lyme disease. Immuno-PCR is a modification of the conventional ELISA format in which the detecting antibody is labeled with a DNA label, and is applicable to the analysis of biological samples (see, e.g., U.S. Pat. No. 5,665,539 and U.S. Patent Application Publication No. 2005/0239108; all herein incorporated by reference). The amplification ability of PCR provides large amounts of the DNA label which can be detected by various methods, typically gel electrophoresis with conventional staining (e.g., Sano et al., Science, 258:120-122, 1992). This method can also include the direct conjugation of the DNA label to the antibody and replacement of gel electrophoresis by using labeled primers to generate a PCR product that can be assayed by ELISA or using real time quantitative PCR. In an example of the real-time PCR method, PCR is used to amplify DNA in a sample in the presence of a nonextendable dual labeled fluorogenic hybridization probe. One fluorescent dye serves as a reporter and its emission spectra is quenched by the second fluorescent dye. The method uses the 5′ nuclease activity of Taq polymerase to cleave a hybridization probe during the extension phase of PCR. The nuclease degradation of the hybridization probe releases the quenching of the reporter dye resulting in an increase in peak emission from the reporter. The reactions are monitored in real time.

Homogeneous immunoassays include, for example, the Enzyme Multiplied Immunoassay Technique (EMIT), which typically includes a biological sample comprising the biomarkers to be measured, enzyme-labeled molecules of the biomarkers to be measured, specific antibody or antibodies binding the biomarkers to be measured, and a specific enzyme chromogenic substrate. In a typical EMIT, excess of specific antibodies is added to a biological sample. If the biological sample contains the molecules to be detected, such molecules bind to the antibodies. A measured amount of the corresponding enzyme-labeled molecules is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the antigen-enzyme complex is antibody-bound makes the EMIT a useful system, enabling the test to be performed without a separation of bound from unbound compounds as is necessary with other immunoassay methods. A homogenous immunoassay, such as an EMIT, can be used to detect any of the molecules associated with a B. burgdorferi-associated condition or disease, such as B. burgdorferi protein antigens listed in Table 3.

Immunoassay kits are also disclosed herein. These kits include, in separate containers (a) monoclonal antibodies having binding specificity for the polypeptides used in the diagnosis of an B. burgdorferi-associated condition/disorder, such as Lyme disease; and (b) and anti-antibody immunoglobulins. This immunoassay kit may be utilized for the practice of the various methods provided herein. The monoclonal antibodies and the anti-antibody immunoglobulins can be provided in an amount of about 0.001 mg to 100 grams, and more preferably about 0.01 mg to 1 gram. The anti-antibody immunoglobulin may also be a polyclonal immunoglobulin, protein A or protein G or functional fragments thereof, which may be labeled prior to use by methods known in the art. In several embodiments, the immunoassay kit includes one, two, three, four or five or more antibodies that specifically bind to molecules associated with a B. burgdorferi-associated condition or disease, such as B. burgdorferi protein antigens listed in Table 3. In embodiments, the antibodies in the kit consist of one, two, three, four or five antibodies that specifically bind to the one, two, three, four or five B. burgdorferi protein antigens listed in Table 3. The immunoassay kit can also include one or more antibodies that specifically bind to one or more of these molecules. Thus, the kits can be used to detect one or more different molecules associated an B. burgdorferi-associated condition, such as Lyme disease.

Immunoassays for polysaccharides and proteins differ in that a single antibody is used for both the capture and indicator roles for polysaccharides due to the presence of repeating epitopes. In contrast, two antibodies specific for distinct epitopes are required for immunoassay of proteins. Exemplary samples include biological samples obtained from subjects including, but not limited to, serum, blood and urine samples. In some examples, an exemplary sample includes bronchoalveolar lavage fluid.

In one particular example, a quantitative ELISA is constructed for detection of at least one of the B. burgdorferi protein antigens listed in Table 3. These immunoassays utilize antibodies, such as mAbs commercially available. Since a polysaccharide is a polyvalent repeating structure, a single mAb may be used for both the capture and indicator phases of an immunoassay. The only requirement is that the mAb have a sufficient affinity. A mAb with an affinity of about 0.5 μM has sufficient affinity.

Capture Device Methods

The disclosed methods can be carried out using a sample capture device, such as a lateral flow device (for example a lateral flow test strip) that allows detection of one or more molecules, such as those described herein.

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept)avidin. The assays are often performed with test strips in which a specific binding pair member is attached to a mobilizable material (such as a metal sol or beads made of latex or glass) or an immobile substrate (such as glass fibers, cellulose strips or nitrocellulose membranes). Particular examples of some of these assays are shown in U.S. Pat. Nos. 4,703,017; 4,743,560; and 5,073,484 (incorporated herein by reference). The test strips include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853; WO 88/08534; and EP-A 0 299 428 (incorporated herein by reference). There are a number of commercially available lateral-flow type tests and patents disclosing methods for the detection of large analytes (MW greater than 1,000 Daltons) as the analyte flows through multiple zones on a test strip. Examples are found in U.S. Pat. No. 5,229,073 (measuring plasma lipoprotein levels), and U.S. Pat. Nos. 5,591,645; 4,168,146; 4,366,241; 4,855,240; 4,861,711; 5,120,643; European Patent No. 0296724; WO 97/06439; WO 98/36278; and WO 08/030546 (each of which are herein incorporated by reference). Multiple zone lateral flow test strips are disclosed in U.S. Pat. Nos. 5,451,504, 5,451,507, and 5,798,273 (incorporated by reference herein). U.S. Pat. No. 6,656,744 (incorporated by reference) discloses a lateral flow test strip in which a label binds to an antibody through a streptavidin-biotin interaction.

In particular examples, the methods disclosed herein include application of a biological sample (such as serum, whole blood or urine) from a human test subject to a lateral flow test device for the detection of one or more molecules (such as one or more molecules associated with Lyme disease, for example, combinations of molecules as described above) in the sample. The lateral flow test device includes one or more antibodies (such as antibodies that bind one or more of the molecules associated with Lyme disease) at an addressable location. In a particular example, the lateral flow test device includes antibodies that bind at least one Lyme disease protein antigen listed in Table 3. The addressable locations can be, for example, a linear array or other geometric pattern that provides diagnostic information to the user. The binding of one or more molecules in the sample to the antibodies present in the test device is detected and the presence or amount of one or more molecules in the sample of the test subject is compared to a control, wherein a change in the presence or amount of one or more molecules in the sample from the test subject as compared to the control indicates that the subject has a B. burgdorferi associated condition, such as Lyme disease.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more molecules disclosed herein. Thus these zones can be viewed as functional sectors or functional regions within the test device.

In general, a fluid sample is introduced to the strip at the proximal end of the strip, for instance by dipping or spotting. A sample is collected or obtained using methods well known to those skilled in the art. The sample containing the particular molecules to be detected may be obtained from any biological source. Examples of biological sources include blood serum, blood plasma, urine, BALF, spinal fluid, saliva, fermentation fluid, lymph fluid, tissue culture fluid and ascites fluid of a human or animal. In a particular example, the biological source is saliva. In one particular example, the biological source is whole blood, such as a sample obtained from a finger prick. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

Another common feature to be considered in the use of assay devices is a means to detect the formation of a complex between an analyte (such as one or more molecules described herein) and a capture reagent (such as one or more antibodies). A detector (also referred to as detector reagent) serves this purpose. A detector may be integrated into an assay device (for example included in a conjugate pad, as described below), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest, for example those described herein).

In other instances, a detector reagent collectively includes an unlabeled first binding partner specific for the analyte and a labeled second binding partner specific for the first binding partner and so forth. Thus, the detector can be a labeled antibody specific for a protein described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab(Fc)) that is present in a secondary capture area.

Flow-Through Device Construction and Design

Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030546. A flow-through device involves a capture reagent (such as one or more antibodies) immobilized on a solid support, typically, a membrane (such as, nitrocellulose, nylon, or PVDF). Characteristics of useful membranes have been previously described; however, it is useful to note that in a flow-through assay capillary rise is not a particularly important feature of a membrane as the sample moves vertically through the membrane rather than across it as in a lateral flow assay. In a simple representative format, the membrane of a flow-through device is placed in functional or physical contact with an absorbent layer (see, e.g., description of “absorbent pad” below), which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample (such as a bodily fluid sample) is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more molecules described herein) can specifically bind to the immobilized capture reagent (such as one or more antibodies). Where detection of an analyte-capture reagent complex is desired, a detector reagent (such as labeled antibodies that specifically bind one or more molecules) can be added with the sample or a solution containing a detector reagent can be added subsequent to application of the sample. If an analyte is specifically bound by capture reagent, a visual representative attributable to the particular detector reagent can be observed on the surface of the membrane. Optional wash steps can be added at any time in the process, for instance, following application of the sample, and/or following application of a detector reagent.

Lateral Flow Device Construction and Design

Lateral flow devices are commonly known in the art. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,168,146; 4,313,734; 4,366,241; 4,435,504; 4,775,636; 4,703,017; 4,740,468; 4,806,311; 4,806,312; 4,861,711; 4,855,240; 4,857,453; 4,861,711; 4,943,522; 4,945,042; 4,496,654; 5,001,049; 5,075,078; 5,126,241; 5,120,643; 5,451,504; 5,424,193; 5,712,172; 6,555,390; 6,258,548; 6,699,722; 6,368,876 and 7,517,699; EP 0810436; EP 0296724; WO 92/12428; WO 94/01775; WO 95/16207; WO 97/06439; WO 98/36278; and WO 08/030546, each of which is incorporated by reference. Further, there are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte.

Many lateral flow devices are one-step lateral flow assays in which a biological fluid is placed in a sample area on a bibulous strip (though non-bibulous materials can be used, and rendered bibulous, e.g., by applying a surfactant to the material), and allowed to migrate along the strip until the liquid comes into contact with a specific binding partner (such as an antibody) that interacts with an analyte (such as one or more molecules) in the liquid. Once the analyte interacts with the binding partner, a signal (such as a fluorescent or otherwise visible dye) indicates that the interaction has occurred. Multiple discrete binding partners (such as antibodies) can be placed on the strip (for example in parallel lines) to detect multiple analytes (such as two or more molecules) in the liquid. The test strips can also incorporate control indicators, which provide a signal that the test has adequately been performed, even if a positive signal indicating the presence (or absence) of an analyte is not seen on the strip.

The construction and design of lateral flow devices is very well known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Lateral flow devices have a wide variety of physical formats that are equally well known in the art. Any physical format that supports and/or houses the basic components of a lateral flow device in the proper function relationship is contemplated by this disclosure.

In some embodiments, the lateral flow strip is divided into a proximal sample application pad, an intermediate test result zone, and a distal absorbent pad. The flow strip is interrupted by a conjugate pad that contains labeled conjugate (such as gold- or latex-conjugated antibody specific for the target analyte or an analyte analog). A flow path along strip passes from proximal pad, through conjugate pad, into test result zone, for eventual collection in absorbent pad. Selective binding agents are positioned on a proximal test line in the test result membrane. A control line is provided in test result zone, slightly distal to the test line. For example, in a competitive assay, the binding agent in the test line specifically binds the target analyte, while the control line less specifically binds the target analyte.

In operation of the particular embodiment of a lateral flow device, a fluid sample containing an analyte of interest, such as one or more molecules described herein (for example, protein antigens listed in Table 1, as discussed above), is applied to the sample pad. In some examples, the sample may be applied to the sample pad by dipping the end of the device containing the sample pad into the sample (such as serum or urine) or by applying the sample directly onto the sample pad (for example by placing the sample pad in the mouth of the subject). In other examples where a sample is whole blood, an optional developer fluid is added to the blood sample to cause hemolysis of the red blood cells and, in some cases, to make an appropriate dilution of the whole blood sample.

From the sample pad, the sample passes, for instance by capillary action, to the conjugate pad. In the conjugate pad, the analyte of interest, such as a protein of interest, may bind (or be bound by) a mobilized or mobilizable detector reagent, such as an antibody (such as antibody that recognizes one or more of the molecules described herein). For example, a protein analyte may bind to a labeled (e.g., gold-conjugated or colored latex particle-conjugated) antibody contained in the conjugate pad. The analyte complexed with the detector reagent may subsequently flow to the test result zone where the complex may further interact with an analyte-specific binding partner (such as an antibody that binds a particular protein, an anti-hapten antibody, or streptavidin), which is immobilized at the proximal test line. In some examples, a protein complexed with a detector reagent (such as gold-conjugated antibody) may further bind to unlabeled, oxidized antibodies immobilized at the proximal test line. The formation of a complex, which results from the accumulation of the label (e.g., gold or colored latex) in the localized region of the proximal test line is detected. The control line may contain an immobilized, detector-reagent-specific binding partner, which can bind the detector reagent in the presence or absence of the analyte. Such binding at the control line indicates proper performance of the test, even in the absence of the analyte of interest. The test results may be visualized directly, or may measured using a reader (such as a scanner). The reader device may detect color or fluorescence from the readout area (for example, the test line and/or control line).

In another embodiment of a lateral flow device, there may be a second (or third, fourth, or more) test line located parallel or perpendicular (or in any other spatial relationship) to test line in test result zone. The operation of this particular embodiment is similar to that described in the immediately preceding paragraph with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

1. Sample Pad

The sample pad is a component of a lateral flow device that initially receives the sample, and may serve to remove particulates from the sample. Among the various materials that may be used to construct a sample pad (such as glass fiber, woven fibers, screen, non-woven fibers, cellosic fibers or paper), a cellulose sample pad may be beneficial if a large bed volume (e.g., 250 μl/cm2) is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

2. Membrane and Application Solution:

The types of membranes useful in a lateral flow device (such as nitrocellulose (including pure nitrocellulose and modified nitrocellulose), nitrocellulose direct cast on polyester support, polyvinylidene fluoride, or nylon), and considerations for applying a capture reagent to such membranes have been discussed previously.

In some embodiments, membranes comprising nitrocellulose are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

3. Conjugate Pad

The conjugate pad serves to, among other things, hold a detector reagent. Suitable materials for the conjugate pad include glass fiber, polyester, paper, or surface modified polypropylene. In some embodiments, a detector reagent may be applied externally, for example, from a developer bottle, in which case a lateral flow device need not contain a conjugate pad (see, for example, U.S. Pat. No. 4,740,468).

Detector reagent(s) contained in a conjugate pad is typically released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and (3-lactose. A mixture of two or more release agents may be used in any given application. In a particular disclosed embodiment, the detector reagent in conjugate pad is a gold-conjugated antibody.

4. Absorbent Pad

The use of an absorbent pad in a lateral flow device is optional. The absorbent pad acts to increase the total volume of sample that enters the device. This increased volume can be useful, for example, to wash away unbound analyte from the membrane. Any of a variety of materials is useful to prepare an absorbent pad, for example, cellulosic filters or paper. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

The following examples are provided to illustrate certain particular features and/or embodiments. These examples should not be construed to limit the disclosure to the particular features or embodiments described.

### EXAMPLE

### Multi-Platform Approach for Microbial Biomarker Identification Using Borrelia burgdorferi as a Model

Successful treatment of many infectious diseases relies on the detection of a pathogen or secreted microbial biomarker early during infection. Early effective treatment is critical to limit damage caused directly by the pathogen or due to the host immune response. A necessary component for diagnosis is selection of an appropriate microbe-specific marker that is indicative of disease (microbial biomarker), or combinations of biomarkers that are present at detectable levels at distinct stages of disease. However, microbes or microbial diagnostic biomarkers contained in patient samples are often at low concentrations during acute infection. While selection of such microbial biomarkers may be done in silico for well characterized bacteria and less genomically complex microbes, like viruses, the prediction of diagnostic biomarkers for bacteria possessing complex genomes and those that undergo antigenic variation are likely to require well-implemented wet lab approaches. Approaches that consider the composition of antigens expressed in vitro often differs from those expressed in vivo.

The clearance of organisms from blood and other accessible biological fluids along with the variable intensity of the immune response to Borrelia burgdorferi biomarkers make the diagnosis and treatment of Lyme disease an ongoing challenge. The difficulties associated with detection of Borrelia burgdorferi made the pathogen an ideal case for developing a multi-platform approach for the detection of a low abundance pathogen from host samples. Analyses of the number of Lyme disease serodiagnostic tests performed at clinical testing centers, and the subsequent results, allowed for an estimate of greater than 300,000 cases of Lyme disease in the U.S. each year. The current method for diagnosis recommended by the CDC is a two-tier serologic assay consisting of an enzyme-linked immunosorbent assay (ELISA) followed by an immunoblot. Administration of the second tier of the test (IgG immunoblot), is not recommended until several weeks post-infection due to its reliance on a detectible IgG antibody response. An IgM immunoblot can be used earlier in disease, with the understanding that the result should not be used solely for diagnosis. Without treatment early during infection, the bacteria may disseminate, leading to the characteristic rheumatologic, cardiac and neurological manifestations of Lyme disease. The clinical features of Lyme disease can be broken down into distinct stages. Early disease is characterized by the tell-tale Erythema Migrans (EM) rash; however, an EM only presents in 60-80% of patients. Early disseminated and late infection phases can be characterized by persistent neurological signs and/or arthritis. Early diagnosis of Lyme disease, leading to the early initiation of treatment, can limit its progression into the late stages of disease and therefore, reduce human morbidity.

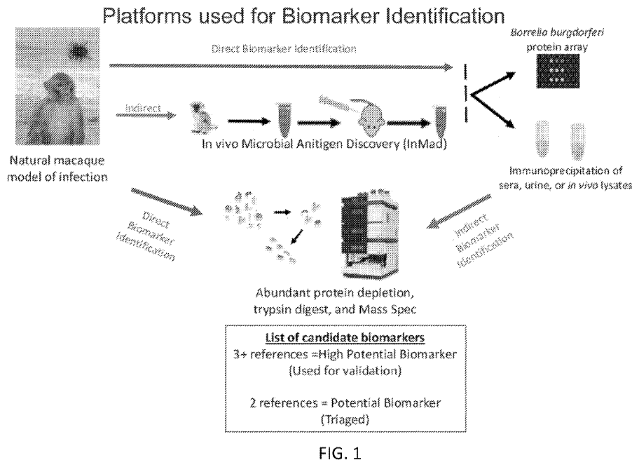

The goal of this study was to develop a standardized approach for identification of microbial antigens that can be detected early during disease and that can be applied to most, if not all infectious diseases. To meet this goal, a discovery-based strategy was designed to identify antigens specific to B. burgdorferi in sera or urine of infected animals. A proteomic approach was selected for the identification of proteins that could be found in samples, proteins were detected either through direct analysis via mass spectrometry (MS) or through indirect analysis, which included an enrichment step using immunoprecipitation prior to MS. Proteomic approaches were used in conjunction with the In vivo Microbial Antigen Discovery (InMAD) platform, in which healthy mice are immunized with filtered serum collected from an infected host (FIG. 1). The InMAD approach was included in the study as it allows for the generation of antibodies in a secondary host to the array of circulating microbial proteins or polysaccharides present at a specific point in an infection of the primary host. Finally, protein arrays were used to validate that the host, either mouse or macaque, had been exposed to an antigen, as well as to begin to map the temporal pattern of biomarker display.

Materials and Methods:

Animals and Infection

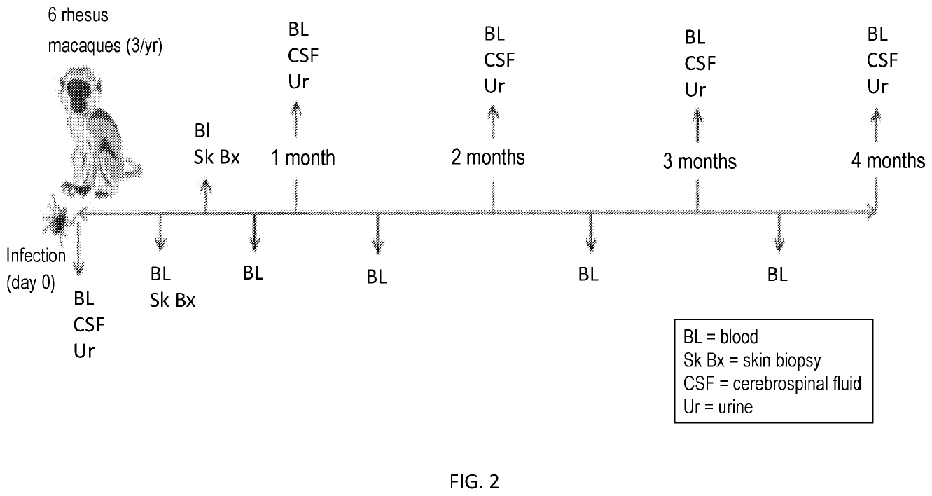

A total of six male rhesus macaques (Macaca mulatta) of Indian origin were used to model human infection. The animals were inoculated with B. burgdorferi strain B31 by feeding infected ticks on them. It was found that 50-90% of ticks feed to repletion with this method, ensuring transmission of the pathogen. The experimental protocol is shown in FIG. 2. To confirm infection, 4 mm skin biopsies taken near the tick feeding sites were obtained at 1- and 2-weeks post-tick feed. Skin samples were subjected to culture and PCR, as described (16, which is hereby incorporated by reference in its entirety). From the blood collected at various time points (see below) serology was performed, using a recently-developed five-antigen test for B. burgdorferi-specific antibodies (Embers et al., Clin Vaccine Immunol, 23(4), 294-303 (2016) doi:10.1128/CVI.00685-15; which is hereby incorporated by reference in its entirety).

Sample Collection

Blood, urine, and CSF were collected throughout a 4-month period following infection (FIG. 2). A 4.9 ml tube of blood was collected at days 0, 7, and then every two weeks for the duration of the study. To preserve proteins in blood, protease inhibitors were introduced to the sample via the BD P100 system (Becton Dickinson) tubes used in collection. The tubes were centrifuged at 1900×g for 10 min. to obtain serum. Urine and CSF were collected at day 0, 1 month, 2 months, 3 months, and 4 months. A protease inhibitor cocktail (cOmplete™, Mini, EDTA-free Protease Inhibitor Cocktail) was made into a 10× stock solution and was added immediately to each urine sample, for a final 1× concentration, transported on ice and then stored at −80° C. At the end of 4 months, animals were euthanized and a gross necropsy was performed in order to obtain tissues in the event that the presence of B. burgdorferi needed to be verified.

Acquisition of Host-Adapted B. burgdorferi Proteins.

The composition of antigens expressed by B. burgdorferi in vitro differs significantly from those expressed in vivo. Therefore, an in vivo culture system was utilized to acquire the proteins expressed by host-adapted spirochetes for analyses. The growth of B. burgdorferi strain B31.5A19 in dialysis membrane chambers (DMCs) that were implanted into rat peritonea was performed as described previously (Akins et al., J Clin Invest, 101(10), 2240-50 (1998) doi:10.1172/JCI2325). The initial quantity of organisms added to each bag was 5×10⁵/ml in a 5-ml volume. Rats were anesthetized by isoflurane gas (1.5 to 2% in oxygen) via nose cone through the entire procedure and received analgesics (buprenorphine subcutaneously at 0.1 mg/kg of body weight) postoperatively. Following implantation of DMCs and suture of rat incisions, organisms were grown for 14 days. Bacterial samples collected from each DMC were counted by dark-field microscopy and samples with the closest concentrations were pooled for processing. Protein lysates were prepared using two methods, protein extraction and sonication, with the purpose of including proteins that may have been diluted out using a single protocol.

For protein extraction, three DMCs were combined to give 1.6×10⁷/ml in a total volume of 13 ml. The spirochetes were pelleted (3,000×g, 30 minutes, without brake), the supernatant was retained and the pellet was washed twice with PBS and resuspended in 1 ml of 50 mM Tris (pH 8)/10 mM EDTA/10% w/v sucrose. This was frozen in a dry ice/methanol bath (˜3 min) and thawed in an ice water bath (approximately 40 minutes). To this, 140 mM NaCl, 1 mM dithiothreitol (DTT) and 0.4 mg/ml lysozyme was added. This was incubated on ice for 45 min. with gentle mixing and subjected to 4 additional freeze/thaw cycles. Cell debris was removed by centrifugation at 18,000×g in a fixed angle rotor. Samples were stored at −80° C. in aliquots. This was also performed with in vitro-cultured B. burgdorferi (1×10⁹ cells total).

To obtain sonicated preparations of B. burgdorferi, samples from individual DMCs with total quantities of spirochetes of 1.83×10⁸ and 1.36×10⁸ were pelleted and frozen for storage. Pellets were defrosted on ice, washed with 10 ml PBS and resuspended in 1 ml PBS on ice. The samples were sonicated with 8 pulses at amplitude 4 for 15 seconds each on ice. Samples were transferred to a microfuge tube and centrifuged for 5 minutes at 13,000 rpm to pellet debris. Protein concentrations were determined with a Nanodrop spectrophotometer (Thermo Fisher Scientific). Samples were stored in aliquots at −20° C.

In Vivo Microbial Antigen Discovery (InMAD)

BALB/c mice were immunized as previously described (Nuti et al., MBio, 2(4) (2011) doi:10.1128/mBio.00136-11). BALB/c mice were selected for the study as they have historically generated an array of antibodies, in high titers, in both InMAD studies as well as for the production of monoclonal antibodies. Briefly, the antigen was prepared by removal of whole microbial cells from the sample. For this experiment frozen serum samples from macaques KD91 and KC92, previously infected with B. burgdorferi, collected at 0, 1, and 2 weeks post-infection, were thawed, centrifuged at 10,000 rpm for 10 minutes followed by filtration through a 0.22 μm syringe filter to eliminate the mass of whole B. burgdorferi cells from the sample. Some cell lysis may have been induced through the removal of bacterial cells. The serum filtrates were then mixed 1:1 with TiterMax Gold Adjuvant and mixed in glass syringes to form an emulsion. Three mice (6-8 weeks old) were immunized via the subcutaneous route with 200 μl of each of the emulsion samples. Due to the limited volume of each sample of macaque serum, a boost of the immunization strategy was not included. Serum was collected from immunized mice, referred to InMAD immune serum, at 0, 4, 6, and 8-weeks post immunization via post retro-orbital bleed. The immune response generated by each mouse was monitored by assessing reactivity with B. burgdorferi whole cell lysates using a standard immunoblot. At 8 weeks post-immunization, the cardiac puncture method was utilized to obtain a final bleed from mice euthanized by extended isoflurane exposure.

Protein Arrays

Initially, the antibody response generated by the infected macaques and immunized mice was gauged using protein array, contracted through Antigen Discovery (Irvine, Ca). Each array was printed with in vitro transcribed and translated open reading frames (orfs) supplemented with recombinant proteins, resulting in an array representing 1397 proteins encoded for by B. burgdorferi. Serum from macaque KD91 collected at 6 weeks post-infection, and the pre-bleed and final bleed (8 weeks post-immunization) from a mouse immunized with serum from macaque KD91 2 weeks post-infection, were used to probe the array. Animal-specific IgG and IgM secondary antibodies were used to identify Ig type. Incubations with antibodies were 1 hour at room temperature.

Nucleic Acid-Programmable Protein Array (NAPPA)

NAPPA is a protein array technology that provides for on-array cell-free protein expression coupled with the capture and display of each protein in defined wells on the array surface. Antibodies found in a serum sample used to probe the array, highlight reactive proteins (Takulapalli et al., High density diffusion-free nanowell arrays. J Proteome Res, 11(8), 4382-91 (2012) doi:10.1021/pr300467q). Each of 10 B. burgdorferi encoded genes (Table 1) included on the NAPPA were selected due to cellular localization. Genes were synthesized by ThermoFisher Scientific in the pENTR221vector and transferred into the pANT7_cGST destination vector. For plasmid preparation, the vectors were transformed into E. coli DH5a and purified by alkaline lysis. For printing, the plasmids were diluted into a Master Mix of printing components including bovine serum albumin, polyclonal anti-tag Ab (goat anti-GST) and a chemical cross-linker (BS-3). Positive controls on the array include Primate IgG and IgM (which confirms secondary reagent activity). Negative controls include empty parent plasmid pANT7_cGST, and Master Mix components without exogenous plasmid. The DNA/Master Mix contents of these 96 well plates are re-arrayed into 384 well plates which are then deposited onto aminosilane-coated silicon nanowell slides using a piezoelectric printing protocol. Printed but unexpressed slides are stored under a dry argon atmosphere, as stability studies have shown that properly stored arrays generate comparable protein signals to freshly printed slides for greater than 8 months after printing. Positive controls on the array include purified primate IgG and IgM, for confirming secondary antibody activity. Negative controls include empty parent plasmid pANT7_cGST (which only produces GST protein alone), and Master Mix components without exogenous plasmid.

TABLE 1
Genes or region included in the limited array.
Each of the sequences with the following Accession
numbers is hereby incorporated by reference as
available on May 29, 2019.

| Gene | Accession Number |
|---|---|
| BB_A68 | NP_045741.1 |
| BB_A64 | NP_045737.2 |
| BB_A74 | NP_045747.1 |
| BB_K32 | AAC66134 |
| vlsE_C6 | atg aag aag gat gat cag att gct |
| region | gct gct att gct ttg agg ggg atg |
| | gct aag gat gga aag ttt gct gtg |
| | aag (SEQ ID NO: 1) |
| BB_A15 | NP_045688 |
| BB_B19 | NP_047005 |
| BB_032 | YP_008686571.1 |
| BB_A24 | NP_045697 |
| BB_0147 | NP_212281.1 |

Arrays were blocked with SuperBlock (Thermo Fisher Scientific) prior to expression to reduce non-specific binding, rinsed with DI water and centrifuged dry. The nano-wells were filled with human cell-free expression system reaction (In Vitro Transcription and Translation coupled system; IVTT; Thermo Fisher Scientific) and a custom micro-reactor device was used for protein expression (Wiktor et al., Sci Rep, 5, 8736 (2015) doi:10.1038/srep08736). After sealing the wells with a polystyrene membrane under pressure (200 PSI), the arrays were incubated for 2 hours at 30° C. for expression and for 0.5 hour at 15° C. for protein capture, and blocked for 30 minutes as above.

The nascent protein arrays were used for serum binding analysis using individual serum samples diluted 1:150 in 5% skim milk in PBS-T. Serum samples were derived from macaques infected with B. burgdorferi. After overnight incubation (14-16 hours) at 4° C. with gentle shaking to ensure even exposure of array surface to sample, the arrays were rinsed and antibody binding was detected with AlexaFlour-647 labeled anti-primate or human IgG (H+L) and 1:200 diluted Cy3 labeled anti-primate or human IgM. The slides were rinsed again to remove unbound secondary antibody, dried by centrifugation and scanned at 635 nm and 535 nm with a Tecan PowerScanner. The resulting images were quantified with the ArrayPro Analyzer Software (Media Cybernetics, Inc.). Data was extracted and median normalized within each subarray. To assure a sufficient margin between positive and negative antibody reactivity a signal-to-noise ratio cutoff of 1.4 was used to identify spots for positive reactivity. This represents greater than 3 standard deviations of the signals above the negative control samples and is a minimal signal-to-noise ratio known to provide detectable signals in ELISA validation assays.

Immunoprecipitation

All immunoprecipitation experiments were carried out using Dynabeads M-270 epoxy (Invitrogen). Antibodies were coupled to 5 mg of beads using the Dynabead antibody coupling kit. For coupling of purified antibodies, 50 μg of antibodies in PBS were coupled. For coupling of antibodies in serum, 150 μl of InMAD immune serum (8 weeks post-immunization) or infected macaque serum (6 and/or 8 weeks post-infection) was used. Antibody-coupled magnetic Dynabeads were used to pull down proteins in either macaque sera or protein lysates from B. burgdorferi adapted to host conditions (culture in DMC). Briefly, antibody-coupled beads were mixed with each sample (a final volume of 250 μl in a binding buffer [50 mM Tris-HCl, 1% Triton X-100, 1 mM EDTA, pH 7.6]) for 4-24 hours rotating at 4° C. and the beads were extracted from the solution using the Dynabead magnet. The beads were washed 4× with PBS. The captured antigens were eluted from the beads in 100 μl of 0.1 M citrate (pH 3.1) rotating 2 minutes at room temperature. The beads were separated out, and proteins in solution were transferred to a clean tube containing 20 μl neutralization buffer (1M Tris, pH 9). Eluted proteins were precipitated and digested for mass spectrometry or separated using SDS-PAGE. Due to limiting volumes of in vivo samples, the use of samples collected from independent macaques at distinct time points (e.g. 1-vs. 2-weeks post-infection) served as controls for immunoprecipitation studies. In that different proteins were identified from IP experiments from each sample, decreasing the likelihood that a protein was pull-downed through non-specific binding.

Sample Preparation for Mass Spectrometry

Macaque sera isolated from each animal at 1- and 2-weeks post-infection and urine from 4 weeks post-infection were analyzed. CSF was not included in the analysis as it is a difficult sample to collect for diagnosis, however it is available for future studies. Samples were prepared for mass spectrometry using either the FASP method for sera or chloroform precipitation for urine samples, followed by trypsin digest. Prior to digestion, serum samples were depleted of the 14 most abundant proteins using the Hu-14 depletion column, per manufacturer's instructions (Agilent) and concentrated using a protein concentrator with a 10 kDa cut off. Samples were prepared for analysis using in-solution digest with DTT, iodoacetamide, and trypsin. Immunoprecipitated proteins were precipitated with a chloroform-methanol extraction prior to reduction, deacetylation, and digestion.

Liquid Chromatography

The trypsin-digested peptides from each sample were analyzed by liquid chromatography-mass spectrometry using a discovery approach at the Nevada Proteomics Center (University of Nevada, Reno). Briefly, peptide mixtures were separated using an UltiMate 3000 RSLCnano system (Thermo Fisher Scientific) on a self-packed UChrom C18 column and eluted using a digital Pico View nanospray source. Mass spectral analysis was performed using an Orbitrap Fusion mass spectrometer (Thermo FisherScientific). For analysis of results, tandem mass spectra were extracted and charge state deconvoluted by Proteome Discoverer version 2.1. All MS/MS samples were analyzed using Sequest and validated using Scaffold (version Scaffold 4.5.1) software. Peptide identification is reported as the X-correlation (cross-correlation value) as reported by the Sequest program.

Results

Infection Status and Serological Response to Exposure with B. burgdorferi

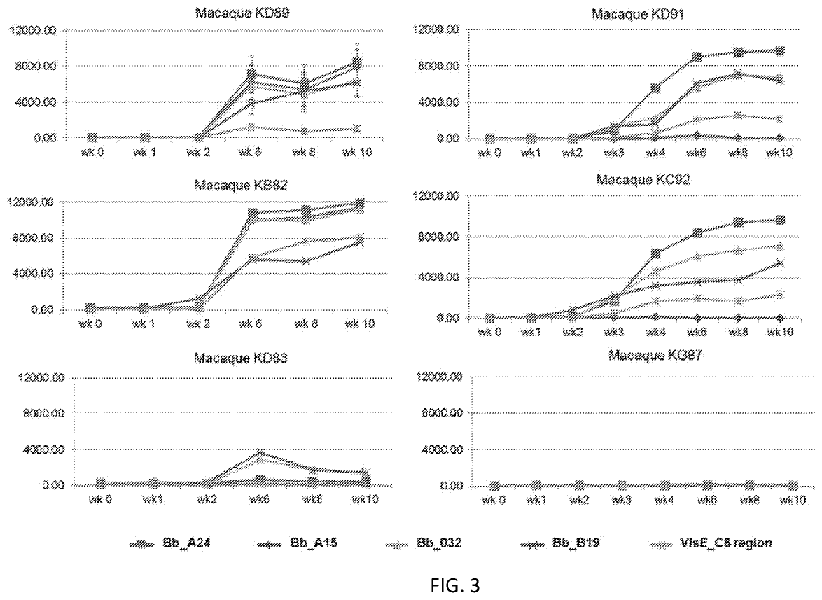

A macaque model of human infection was implemented to study the presence of microbial biomarkers in the host, as well as the host immune response to infection with the Lyme disease spirochete. This model was chosen because the disease process and variability in immune responses reflects those seen in humans. Skin biopsy was taken from each macaque near a tick bite site papule or patch of erythema. Analyses of the skin punches indicated that 5/6 macaques were biopsy-positive for B. burgdorferi (Table 2). The longitudinal serological response to OspC, OspA, DbpA, OppA2, and the C6 peptide of VlsE were assessed with a 5-antigen multiplex IgG assay (FIG. 3). Over the six-week monitoring period 4/6 macaques developed an immune response that increased over time to at least 4 of the 5 antigens, and 5/6 macaques developed a response to 2 or more antigens. While 100% correlation was not seen between the biopsy and serological responses, the results indicate that all 6 animals initially developed an infection with B. burgdorferi.

TABLE 2
Clinical determinants of macaques infected using the tick-bite model of infection. Including
number of infected ticks removed from each macaque and infection status of the animals as determined by
skin biopsy followed by PCR or culture.

| | Skin Biopsy- | Skin Biopsy- | Ticks removed |
|---|---|---|---|
| | PCR | culture | (post-feeding) |
| KD91 | + | − | 7 |
| KC92 | + | − | 14 |
| KG87 | + | − | 8 |
| KB82 | − | − | 13 |
| KB83 | + | − | 5 |
| KD89 | − | + | 6 + 4 partial |

Temporal Accumulation of Antibodies to Borrelia-Specific Antigens

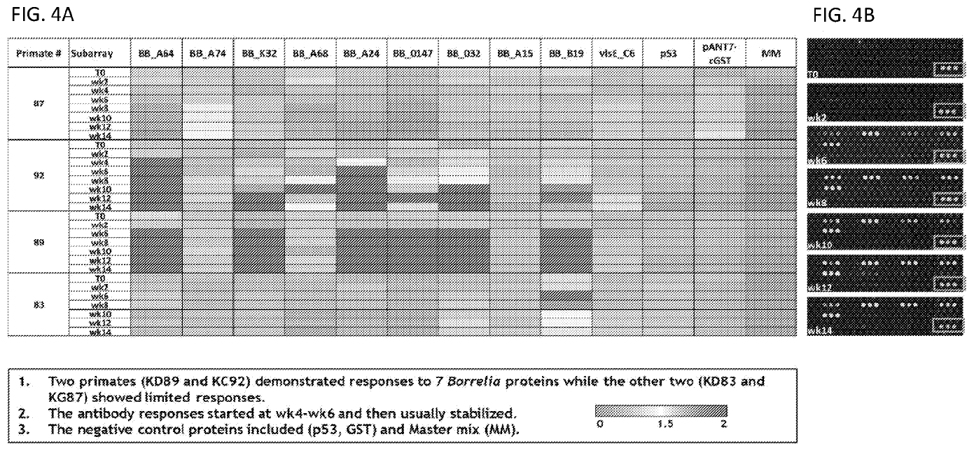

As a supplemental approach to assess the temporal pattern of antibody generation to Borrelia in macaques, a 10 protein B. burgdorferi-specific NAPPA array was developed. Protein selection (Table 1) for this limited array was based on protein localization (outer membrane) and interest as a diagnostic antigen. The array was probed with serum samples collected throughout the infection of macaques. The data indicate that a subset of the macaques developed an immune response to 7/10 B. burgdorferi proteins. The temporal pattern of the response was overlapping, but not constant between the animals (FIG. 4). Importantly, a detectible response was not recorded from samples collected from macaque KG87 using either the 5-antigen multiplexed assay or the NAPPA (FIGS. 3 and 4), indicating the animal remained seronegative throughout the sampling period. The infection status of this animal at the study end point was not evaluated. The variable pattern between macaques is a trend that is consistent with infection patterns in patients, as evident in the variability of the results from the current two-tier assay (i.e. 5 or more of the 10 proteins on the Western blot are required for a positive result). The C6 reactivity by 4 of the 6 monkeys is apparent when the Luminex-based assay is performed, but was not detected but the NAPPA array. It is possible this was due to the nature of the antigen—peptide versus protein and how it is presented in each assay system.

Direct Identification of Biomarkers

Sera and urine samples from infected macaques were submitted for analysis using a discovery approach to mass spectrometry. InMAD immune serum collected from mice was not submitted for mass spectrometry as microbial biomarkers at low concentrations in the macaque serum or urine would be diluted in the InMAD immune sera, further minimizing the chance of detection. Analyses of the serum samples resulted in identification of six antigens that met the criteria of potential biomarkers, two of which were detected late in infection by the 5-antigen multiplex serological assay; DbpA and VlsE (Table 3). In addition, two antigens, Fla and p83/100, defined as markers of the two-tier (immunoblot) assay were detected, suggesting that there is overlap between our strategy and established benchmarks (Dressler et al., J Infect Dis, 167(2), 392-400 (1993); and Ryffel et al., Clin Microbiol Infect, 4(4), 205-212 (1998)). Inclusion as a potential biomarker necessitated 2 or more identifications. Biomarkers identified through this small sample will be validated in a larger panel of macaque samples along with acute patient samples prior to inclusion in a diagnostic.

Burrelia burgdorferi anitgens detected in infected macaque serum samples
using a combination of mass spectrometry and protein array. A range in X-correlation
values reflects identification from multiple samples. X-correlation value is an indication of
the alignment of the peptide detected with the predicted mass-to-charge ratio of the theoretical
peptide as assessed by Sequest (cut-off for inclusion 1.8). The array reactivity ranking was a
reflection of the florescent intensity on the array, sample type indicates if macaque sera or
InMAD immune sera was used to probe the array.

| | | | MS X Correlation |
|---|---|---|---|
| | | | Value or Array |
| Name; | | | Activity Ranking |
| Accession No. | Method | Sample | (sample type) |
| DpbA | Direct MS | MacaqueKD91-1 and 2 week post | Range: 1.86-4.34 |
| (BB_A24; | | infection | |
| NP_045697) | | | |
| Q1W5I8 | | | |
| DpbA | Protein Array | MacaqueKD91-6 week post | IgG #10 (macaque) |
| (BB_A24; | | infection | |
| NP 045697) | | | |
| Q1W5I8 | | | |
| Fla (BB_0147; | IP/MS | InMad Immune sera KD91 | Range: 1.83-5.90 |
| NP_212281.1) | | immunized (x3)-in vivo lysates; | |
| | | KD89T6 coupled-IP of in vivo | |
| Primary (citable) | | | |
| accession | | | |
| number: P11089 | | | |
| Secondary | | | |
| accession | | | |
| number(s): | | | |
| O31322, | | | |
| P15295, Q44938 | | | |
| Fla (BB_0147; | Direct MS | Macaque KD89-1week post | 2.79 |
| NP_212281.1) | | infection | |
| Primary (citable) | | | |
| accession | | | |
| number: P11089 | | | |
| Secondary | | | |
| accession | | | |
| number(s): | | | |
| O31321 | | | |
| P15295, Q44938 | | | |
| Fla (BB_0147 | Protein Array | InMad immune sera-8 week post | IgM #5 (mouse) |
| NP_212281.1) | | immunization | |
| Primary (citable) | | | |
| accession | | | |
| number: P11089 | | | |
| Secondary | | | |
| accession | | | |
| riumber(s): | | | |
| O31322, | | | |
| P15295, Q44938 | | | |
| VlsE | IP/MS | Macaque KD89 antibodies coupled | 2.36 |
| (BB_F0041) | | to beads-IP of macaque | |
| G5IXI6 | | KD89 serum-2-week post - | |
| | | infection | |
| VlsE | Direct MS | Macaque KG87-1 week post | 2.62 |
| (BB_F0041) | | infection | |
| G-5IXI6 | | | |
| VlsE | Protein Array | Macaque KD91- 6 week post | IgG #21 (macaque) |
| (BB_F0041) | | infection | IgM #33 (macaque) |
| G5IXI6 | | | |
| p83/100 | Direct MS | Macaque KD91-1 and 2 weeks | 2.45 |
| (BB_0744) | | post-infection (pooled) | |
| A0A0H3LMW4 | | | |
| p83/100 | Protein Array | InMad immune sera-8 weeks post | IgM #7 (mouse) |
| (BB_0744) | | immunization | |
| A0A0H3LMW4 | | | |
| BB_G31 | IP/MS | InMad immune sera coupled to | 2.19 |
| AAC66060 | | beads-IP of in vivo lysate | |
| BB_G31 | Direct MS | Macaque KD91-1 and 2 week | 2.51 |
| AAC66060 | | post-infection (pooled) | |
| BB_J48 | Direct MS | Macaque KD91-1 and 2 week | Range 2.22-2.35 |
| AAC66130 | | post-infection (not pooled) | |

Indirect Identification of Biomarkers

Proteins secreted or shed by B. burgdorferi in the serum or urine of the host may be at a concentration below the limit of detection by mass spectrometry. In order to enhance the prospect of detecting the proteins by mass spectrometry, immunoprecipitation was utilized to enrich samples for antigenic biomarkers. A key aspect of the InMAD process is that it allows for the generation of a diverse array of antibodies to biomarkers found early in infection. To increase the diversity of proteins that were isolated from infected animal samples and the host-adapted bacterial protein lysates (DMC-cultured), antibodies generated by mice in the InMAD immune sera, as well as by macaques at 6 weeks post-infection were used as receptors in immunoprecipitation experiments. By adding the immunoprecipitation step, an additional biomarker of interest was identified from a macaque sample that had already been detected by direct MS (VlsE; Table 3). In addition, proteins from host-adapted bacterial lysate (DMC-cultured) were enriched for immunogenic proteins, prior to identification by MS, resulting in the identification of two proteins of interest (Fla and BB G31). It is important to note that DMC-cultured spirochetes are protected from the immune response, so antigens involved in immune evasion may be differentially expressed in this system.

Identification of Antibodies Generated to Borrelia burgdorferi

A whole genome proteome array, produced and probed by Antigen Discovery, was utilized for initial studies, to detect immunogenic proteins in macaque and murine hosts. Each of the 1397-in-vitro transcribed and translated genes on the array were ranked by fluorescence intensity generated upon probing with each sample. While data generated using the array is limited, using serum samples from a single macaque and one mouse from the InMAD experiment to probe the array, the data is included to support the mass spec results. As such, the data were considered as a single factor in our multi-platform approach to establish target antigens present in a model of B. burgdorferi infection (Table 3).

### Discussion

The gold standard tests for detection of many infectious diseases require that samples are sent to a central or specialty laboratory for culture and/or detection assay, processes that can take days to weeks for a definitive diagnosis. Furthermore, samples with a low bioburden may drive a false-negative-results without an amplification step, thereby adding additional time from sample collection to results. The CDC recommended assay for laboratory diagnosis of Lyme disease is a two-step serology-based assay, which requires the development of a prescribed immune response. The laboratory diagnosis is often considered secondary to patient history, including exposure of tick habitats. Disease diagnosis that is dependent upon the patient developing an immune response is problematic for multiple reasons, as i) development of an antibody response can delay treatment by several weeks, ii) the nature of seronegative patients would necessitate additional testing strategies, and iii) serological assays do not distinguish between new and previously treated infections. These are among the reasons that a sensitive, defined antigen-based assay for early detection of many diseases is needed, with the inhibiting factor being the discovery of microbial biomarkers in patient samples that are within the level of detection of established assays. Minimal concentration of biomarkers early in infection may necessitate sample enrichment for successful biomarker discovery. Throughout the course of this study different enrichment strategies were used to identify microbial biomarkers, examples of enrichment are as follows i.) concentration of all biomarkers (concentration of host and microbial markers in urine), ii.) enrichment of microbial biomarkers (depletion of host proteins from serum), and iii.) enrichment of specific biomarker (immunoprecipitation). While each of these techniques may lead to loss of some targets, an experimental design that allows for data collection using overlapping approaches will minimize the loss. Beyond enrichment, the problem of low target concentration, as well as that of variation of biomarkers between patients, can be addressed through the inclusion of multiple microbial biomarkers in the development of a sensitive and specific diagnostic for early detection. The format of such multiplexed assays will be defined by the target limit of detection and adaptability to clinical workflow.

As a proof-of-principle, multiple platforms were exploited in an effort to unmask B. burgdorferi biomarkers that may have been missed in single step methods due to difference in concentrations of host versus microbial proteins. By limiting samples used for both direct analysis and in the InMAD studies (which defines the immune response to circulating antigens at a specific point in time) to those isolated early in infection, only biomarkers that promote an early diagnosis should have been identified. A recent study by the Turko group, with a similar goal of identifying biomarkers for Lyme disease, focused on identifying biomarkers found to be abundant in B. burgdorferi B31 cultured in vitro, in patient samples using MS. Their studies found that peptides from the OspA could be detected in early patient serum samples upon concentration, but not in samples collected later in infection. OspA, a potential biomarker that was not highlighted in the present study was also found in urine samples concentrated by Nanotrap. These reports confirm the idea presented here that in order detect low-abundance B. burgdorferi proteins, sample concentration is critical.

Samples used in this study were based upon a well-defined model of disease that closely mimics Lyme disease in humans, namely the macaque model of infection by tick vector, which was combined with the temporally defined InMAD assay. A conservative approach to biomarker identification was taken by defining hypothetical target antigens as those that were identified using more than a single technique or in multiple samples. More specifically, proteins that were identified more than once were classified as potential biomarkers, and those identified three or more times were classified as high-potential biomarkers. The data generated using the platform was integrated to identify six proteins that were detected as candidate early microbial indicators of infection. Target biomarkers present in serum from the infected host included both targets previously discussed as diagnostic antigens as well as those that are not normally considered candidates for use as a diagnostic of Lyme disease, opening new avenues of research. Furthermore, more emphasis was placed on serum than urine samples, allowing for the possibility that additional microbial biomarkers may be present in urine.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A kit for detecting a B. burgdorferi-associated condition, comprising at least one molecule capable of detecting at least one B. burgdorferi-associated molecule presented herein, such as Table 3, and directions for using the kit to detect a B. burgdorferi-associated condition.

2. The kit of claim 1, wherein the kit is one for self monitoring and the at least one molecule capable of detecting at least one B. burgdorferi-associated molecule presented in herein, such as Table 3, is presented on a test strip.

3. The kit of claim 2, wherein the test strip is a dipstick test strip.

4. The kit of claim 2 or claim 3, wherein the kit includes directions for using the kit to diagnose a subject with Lyme disease.

5. The kit of any one of claims 2-4, wherein the kit includes directions for using the kit to monitor efficacy of a therapy.

6. A method of identifying a diagnostic indicator for a B. burgdorferi-associated condition or disease, comprising:
immunizing a veterinary subject which is not afflicted with the B. burgdorferi-associated condition or disease with a human biological sample obtained from a human subject having the B. burgdorferi-associated condition or disease to generate antibodies;
collecting a biological sample comprising the generated antibodies from the immunized veterinary subject; and
identifying one or more diagnostic indicators for the selected condition or disease, wherein an alteration in at least one antigen detected by the generated antibodies in the biological sample obtained from the immunized veterinary subject relative to the control indicates that such antigen is a diagnostic indicator for the B. burgdorferi-associated condition or disease.

7. The method of claim 6, wherein the biological sample from the immunized veterinary subject is a serum sample.

8. The method of claim 6, wherein the biological sample from the immunized veterinary subject is a whole blood sample.

9. The method of any one of claims 6-8, further comprising obtaining the human biological sample from the human subject with the B. burgdorferi-associated condition or disease.

10. The method of any one of claims 6-9, wherein the human biological sample is serum or blood sample.

11. The method of any one of claims 6-9, wherein the human biological sample is a urine sample.

12. The method of claim 6, wherein the immunized veterinary subject biological sample is urine.

13. The method of any one of claims 6-12, wherein identifying one or more diagnostic indicators for the B. burgdorferi-associated condition or disease comprises using one-dimensional or two-dimensional immunoblots.

14. The method of any one of claims 6-12, wherein identifying one or more diagnostic indicators for the B. burgdorferi-associated condition or disease comprises using one-dimensional or two-dimensional immunoblots followed by mass spectroscopy.

15. The method of anyone of claims 6-14 wherein the B. burgdorferi-associated condition or disease, is Lyme disease.

16. A method of diagnosing a subject with Lyme disease or monitoring the efficacy of a therapy for Lyme disease, comprising:
detecting at least two B. burgdorferi-associated molecule in a sample obtained from a subject exhibiting one or more signs or symptoms associated with Lyme disease or a subject known to be at risk of acquiring Lyme disease, wherein the at least two B. burgdorferi-associated molecules are at least two antigens listed in Table 3, thereby diagnosing the subject with Lyme disease or monitoring the efficacy of the therapy for Lyme disease.

17. The method of claim 16, further comprising comparing detection of the at least two B. burgdorferi-associated molecules in the sample obtained from the subject exhibiting one or more signs or symptoms associated with Lyme disease to a control, wherein increased detection of the at least two B. burgdorferi-associated molecules relative to a control indicates that the subject has Lyme disease.

18. The method of claim 17, wherein detecting of that at least two B. burgdorferi-associated molecules comprises usage of at least two antibodies specific for the at least two B. burgdorferi-associated molecule.

19. The method of any one of claims 16-18, wherein the method is used for diagnosing a subject with Lyme disease.

20. The method of any one of claims 16-18, wherein the method is used for monitoring the efficacy of therapy of Lyme disease.
